(19) United States
(12) Patent Application Publication
(10) Pub. No.: US 2012/0253141 A1
(43) Pub. Date: Oct. 4, 2012

(54) METHODS AND SYSTEMS FOR PASSIVE PHOTOPLETHYSMOGRAPH SENSING

(75) Inventors: Paul Stanley Addison, Edinburgh (GB); Keith Manning, Linlithgow (GB); James N. Watson, Dunfermline (GB)

(73) Assignee: Nellcor Puritan Bennett Ireland, Galway (IE)

(21) Appl. No.: 13/076,895

(22) Filed: Mar. 31, 2011

(51) Int. Cl. A61B 6/00; G01J 1/44

(52) U.S. Cl. 600/301; 250/214 R

(57) ABSTRACT

Systems and methods are provided for passive photoplethysmograph sensing. A patient monitoring system may provide active sensing, passive sensing, or both. In some cases, a patient monitor may determine whether to provide passive or active sensing. Passive photoplethysmograph sensing may be used to determine physiological information such as pulse rate, respiration rate, or other information. Passive photoplethysmograph sensing may allow for reduced power consumption relative to active sensing.

### SUMMARY

Systems and methods are provided for providing passive photoplethysmograph (PPG) sensing (“passive sensing”). PPG sensors may, but need not, include an explicit light source such as a light emitting diode (LED) or other emitter. A passive photonic signal, generated without an activated explicit light source, may be received by one or more photodetector or arrays of photodetectors. Physiological information such as respiration rate, pulse rate, or other physiological information may be derived at least in part from the received passive photonic signal. In some embodiments, the passive photonic signal may be optically filtered (e.g., spectrally filtered using optical filters) or otherwise attenuated by the patient monitoring system prior to being received by the photodetector.

In some embodiments, a patient monitoring system may determine whether to provide active or passive PPG sensing. This determination may be based on, for example, signal metrics such as signal to noise ratio, a value of a physiological parameter, changes in a physiological parameter, any other suitable information, or any combination thereof. For example, a patient monitoring system may provide passive sensing. If it is determined, for example, that the signal to noise ratio of a PPG signal based at least in part on a passive photonic signal has fallen below a threshold, the patient monitoring system may determine that active sensing is to be provided. The patient monitoring system may provide active sensing by activating one or more explicit light sources. The patient monitoring system may provide passive sensing by deactivating, or not activating, one or more explicit light sources.

### BRIEF DESCRIPTION OF THE DRAWINGS

The above and other features of the present disclosure, its nature and various advantages will be more apparent upon consideration of the following detailed description, taken in conjunction with the accompanying drawings in which:

FIG. 1 shows an illustrative patient monitoring system in accordance with an embodiment;

FIG. 2 is a block diagram of the illustrative patient monitoring system of FIG. 1 coupled to a patient in accordance with an embodiment;

FIG. 3 is a block diagram of an illustrative sensor with an activated light source in accordance with an embodiment;

FIG. 4 is a block diagram of an illustrative sensor with an unactivated light source in accordance with an embodiment;

FIG. 5 is a block diagram of an illustrative sensor without an explicit light source in accordance with an embodiment;

FIG. 6 is a block diagram of an illustrative sensor with an optical filter in place in accordance with an embodiment;

FIG. 7 is a block diagram of an illustrative signal processing system in accordance with an embodiment;

FIG. 8 is an illustrative signal which may be analyzed in accordance with an embodiment;

FIG. 9 is a flow diagram of illustrative steps for determining physiological information in accordance with an embodiment;

FIG. 10 is a flow diagram of illustrative steps for determining whether to provide active or passive sensing in accordance with an embodiment;

FIG. 11 is a flow diagram of illustrative steps for determining whether to provide passive sensing in accordance with an embodiment;

FIG. 12 is a flow diagram of illustrative steps for determining whether to provide active sensing in accordance with an embodiment;

FIG. 13 is a flow diagram of illustrative steps for controlling the output of an explicit light source in accordance with an embodiment; and

FIG. 14 is a flow diagram of illustrative steps for determining physiological information in accordance with an embodiment.

### DETAILED DESCRIPTION

A plethysmograph may include any suitable device or system for measuring changes within organs of the body, while a photoplethysmograph (PPG) specifically relies on optical (i.e., photonic) measurements. Passive PPG sensing (“passive sensing”) refers to sensing using a photodetector of a PPG sensor without an activated emitter (e.g., an explicit photonic source). In some embodiments, an emitter such as an LED may be de-activated or “turned off” while a photodetector is used to detect electromagnetic radiation attenuated by a patient. In some embodiments, a passive PPG sensor need not include an emitter.

An oximeter is a medical device that may determine the oxygen saturation of the blood. One common type of oximeter is a pulse oximeter, which may indirectly measure the oxygen saturation of a patient's blood (as opposed to measuring oxygen saturation directly by analyzing a blood sample taken from the patient). Pulse oximeters may be included in patient monitoring systems that measure and display various blood flow characteristics including, but not limited to, the oxygen saturation of hemoglobin in arterial blood. Patient monitoring systems may also measure and display additional physiological parameters, such as a patient's pulse rate and blood pressure.

An oximeter may include a light sensor that is placed at a site on a patient, typically a fingertip, toe, forehead or earlobe, or in the case of a neonate, across a foot. The oximeter may use an explicit light source to pass light through blood perfused tissue and photoelectrically sense the absorption of the light in the tissue. In addition, locations which are not typically understood to be optimal for pulse oximetry serve as suitable sensor locations for the blood pressure monitoring processes described herein, including any location on the body that has a strong pulsatile arterial flow. For example, additional suitable sensor locations include, without limitation, the neck to monitor carotid artery pulsatile flow, the wrist to monitor radial artery pulsatile flow, the inside of a patient's thigh to monitor femoral artery pulsatile flow, the ankle to monitor tibial artery pulsatile flow, and around or in front of the ear. Suitable sensors for these locations may include sensors for sensing absorbed light based on detecting reflected light. In all suitable locations, for example, the oximeter may measure the intensity of light that is received at the light sensor as a function of time. The oximeter may also include sensors at multiple locations. A signal representing light intensity versus time or a mathematical manipulation of this signal (e.g., a scaled version thereof, a log taken thereof, a scaled version of a log taken thereof, etc.) may be referred to as the photoplethysmograph (PPG) signal. In addition, the term “PPG signal,” as used herein, may also refer to an absorption signal (i.e., representing the amount of light absorbed by the tissue) or any suitable mathematical manipulation thereof. The light intensity or the amount of light absorbed may then be used to calculate any of a number of physiological parameters, including an amount of a blood constituent (e.g., oxyhemoglobin) being measured as well as a pulse rate and when each individual pulse occurs.

In some applications, the light passed through the tissue is selected to be of one or more wavelengths that are absorbed by the blood in an amount representative of the amount of the blood constituent present in the blood. The amount of light passed through the tissue varies in accordance with the changing amount of blood constituent in the tissue and the related light absorption. Red and infrared (IR) wavelengths may be used because it has been observed that highly oxygenated blood will absorb relatively less Red light and more IR light than blood with a lower oxygen saturation. By comparing the intensities of two wavelengths at different points in the pulse cycle, it is possible to estimate the blood oxygen saturation of hemoglobin in arterial blood.

When the measured blood parameter is the oxygen saturation of hemoglobin, a convenient starting point assumes a saturation calculation based at least in part on Lambert-Beer's law. The following notation will be used herein:

I(λ,t)=Iₒ(λ)exp(−(sβₒ(λ)+(1−s)βᵣ(λ))l(t)) (1)

where:
λ=wavelength;
t=time;
I=intensity of light detected;
I₀-intensity of light transmitted;
s=oxygen saturation;
β₀,βᵣ=empirically derived absorption coefficients; and
l(t)=a combination of concentration and path length from emitter to detector as a function of time.

The traditional approach measures light absorption at two wavelengths (e.g., Red and IR), and then calculates saturation by solving for the “ratio of ratios” as follows.

1. The natural logarithm of Eq. 1 is taken (“log” will be used to represent the natural logarithm) for IR and Red to yield

log I=log Iₒ−(sβₒ+(1−s)βᵣ)l (2)

2. Eq. 2 is then differentiated with respect to time to yield

$$\frac{d log I}{d t} = - ( s {β}_{o} + ( 1 - s ) {β}_{r} ) \frac{d l}{d t} . \qquad ( 3 )$$

3. Eq. 3, evaluated at the Red wavelength λ_{R}, is divided by Eq. 3 evaluated at the IR wavelength λ_{IR} in accordance with

$$\frac{d log I ( {λ}_{R} ) / d t}{d log I ( {λ}_{IR} ) / d t} = \frac{s {β}_{o} ( {λ}_{R} ) + ( 1 - s ) {β}_{r} ( {λ}_{R} )}{s {β}_{o} ( {λ}_{IR} ) + ( 1 - s ) {β}_{r} ( {λ}_{IR} )} . \qquad ( 4 )$$

4. Solving for s yields

$$s = \frac{\frac{d log I ( {λ}_{IR} )}{d t} {β}_{r} ( {λ}_{R} ) - \frac{d log I ( {λ}_{R} )}{d t} {β}_{r} ( {λ}_{IR} )}{\begin{matrix}\frac{d log I ( {λ}_{R} )}{d t} ( {β}_{o} ( {λ}_{IR} ) - {β}_{r} ( {λ}_{IR} ) ) - \\ \frac{d log I ( {λ}_{IR} )}{d t} ( {β}_{o} ( {λ}_{R} ) - {β}_{r} ( {λ}_{R} ) )\end{matrix}} . \qquad ( 5 )$$

5. Note that, in discrete time, the following approximation can be made:

$$\frac{d log I ( λ , t )}{d t} ≃ log I ( λ , {t}_{2} ) - log I ( λ , {t}_{1} ) . \qquad ( 6 )$$

6. Rewriting Eq. 6 by observing that log A−log B=log(A/B) yields

$$\frac{d log I ( λ , t )}{d t} ≃ log ( \frac{I ( {t}_{2} , λ )}{I ( {t}_{1} , λ )} ) . \qquad ( 7 )$$

7. Thus, Eq. 4 can be expressed as

$$\frac{\frac{d log I ( {λ}_{R} )}{d t}}{\frac{d log I ( {λ}_{IR} )}{d t}} ≃ \frac{log ( \frac{I ( {t}_{1} , {λ}_{R} )}{I ( {t}_{2} , {λ}_{R} )} )}{log ( \frac{I ( {t}_{1} , {λ}_{IR} )}{I ( {t}_{2} , {λ}_{IR} )} )} = R , \qquad ( 8 )$$

where R represents the “ratio of ratios.”
8. solving Eq. 4 for s using the relationship of Eq. 5 yields

$$s = \frac{{β}_{r} ( {λ}_{R} ) - R {β}_{r} ( {λ}_{IR} )}{R ( {β}_{o} ( {λ}_{IR} ) - {β}_{r} ( {λ}_{IR} ) ) - {β}_{o} ( {λ}_{R} ) + {β}_{r} ( {λ}_{R} )} . \qquad ( 9 )$$

9. From Eq. 8, R can be calculated using two points (e.g., PPG maximum and minimum), or a family of points. One method applies a family of points to a modified version of Eq. 8. Using the relationship

$$\frac{d log I}{d t} = \frac{d I / d t}{I} , \qquad ( 10 )$$

Eq. 8 becomes

$$\begin{matrix}\frac{\frac{d log I ( {λ}_{R} )}{d t}}{\frac{d log I ( {λ}_{IR} )}{d t}} ≃ \frac{\frac{I ( {t}_{2} , {λ}_{R} ) - I ( {t}_{1} , {λ}_{R} )}{I ( {t}_{1} , {λ}_{R} )}}{\frac{I ( {t}_{2} , {λ}_{IR} ) - I ( {t}_{1} , {λ}_{IR} )}{I ( {t}_{1} , {λ}_{IR} )}} \\ = \frac{[ I ( {t}_{2} , {λ}_{R} ) - I ( {t}_{1} , {λ}_{R} ) ] I ( {t}_{1} , {λ}_{IR} )}{[ I ( {t}_{2} , {λ}_{IR} ) - I ( {t}_{1} , {λ}_{IR} ) ] I ( {t}_{1} , {λ}_{R} )} \\ = R ,\end{matrix} \qquad ( 11 )$$

which defines a cluster of points whose slope of y versus x will give R when

x=[I(t₂,λ_{IR})−I(t₁,λ_{IR})]I(t₁,λ_{R}), (12)

and

y=[I(t₂,λ_{R})−I(t₁,λ_{R})]I(t₁,λ_{IR}). (13)

Once R is determined or estimated, for example, using the techniques described above, the blood oxygen saturation can be determined or estimated using any suitable technique for relating a blood oxygen saturation value to R. For example, blood oxygen saturation can be determined from empirical data that may be indexed by values of R, and/or it may be determined from curve fitting and/or other interpolative techniques.

The term “explicit light source” as user herein shall refer to a device which by design supplies photons of particular and substantially predictable properties. An emitter, such as a laser or LED, may be an explicit light source. The term “ambient light” as used herein shall refer to diffuse terrestrial light including scattered, reflected, or otherwise attenuated and substantially diffuse sunlight, as well as any other terrestrial source of photons other than an explicit light source, such as lamps, displays, thermal radiation from objects, or any combination thereof.

In some embodiments, an explicit light source need not be used to provide a photonic signal for physiological indications. For example, in some embodiments, thermal radiation emitted from a subject may be detected by a suitable photodetector. In a further example, ambient light attenuated (e.g., scattered, absorbed, transmitted, reflected) by a subject may be detected by a suitable photodetector.

The following notation will be used herein:

a=absorptivity;
ε=emissivity;
λ=wavelength;
i_{λ}=spectral intensity (similar to I(λ));
L=path length;
σ=scattering coefficient;
ω=solid angle;
ωᵢ=solid angle of incident intensity;
Φ=phase function;
in which the subscripts are designated as follows: λ refers to a spectral quantity, a refers to absorptive phenomenon, b refers to blackbody radiative properties, ε refers to emissive, i refers to incident, s− refers to scattering away of intensity, and s+ refers to incoming scattered intensity.

The differential change in spectral intensity i_{λ} along some path length L within a scattering and absorbing media at a particular wavelength λ may be formulated as

$$\frac{d {i}_{λ}}{d L} = \frac{d {i}_{λ , a}}{d L} + \frac{d {i}_{λ , e}}{d L} + \frac{d {i}_{λ , s -}}{d L} + \frac{d {i}_{λ , s +}}{d L} , \qquad ( 14 )$$

in which each term of Eq. 14 may be formulated as;

$$\begin{matrix}\frac{d {i}_{λ , a}}{d L} = - {a}_{λ} ( L ) {i}_{λ} ( L ) , & ( 15 ) \\ \frac{d {i}_{λ , e}}{d L} = {ɛ}_{λ} ( L ) {i}_{λ b} ( L ) , & ( 16 ) \\ \frac{d {i}_{λ , s -}}{d L} = - {σ}_{s λ} ( L , ω ) {i}_{λ} ( L , ω ) , \\ and & ( 17 ) \\ \frac{d {i}_{λ , s +}}{d L} = \frac{1}{4 π} {\int }_{0}^{4 π} {σ}_{s λ} ( L , {ω}_{i} ) {i}_{λ} ( L , {ω}_{i} ) {Φ}_{λ} ( L , ω , {ω}_{i} ) d {ω}_{i} . & ( 18 )\end{matrix}$$

The expression shown in Eq. 14 may be referred to as the “equation of transfer” for radiation within a participating media. The four terms on the right hand side of Eq. 14 represent (from left to right) effects from absorption (see Eq. 15), emission (see Eq. 16), scattering loss (see Eq. 17), and scattering gain from all directions (see Eq. 18), respectively. Each term of Eq. 14 may also depend on time (e.g., temporally changing optical properties or sources), location, local temperature, or any other suitable parameter.
Integration of Eq. 14 along a suitable path (e.g., a path through a portion of a subject), along with any suitable boundary conditions and surface phenomena (e.g., reflection), may provide the spectral intensity incident to a photoactive region of a photodetector. It will be understood that Eqs. 14-18 are illustrative, and that any suitable mathematical formalism, simplification, approximation, empirically derived correlation, or other expression may be used to quantify electromagnetic radiation incident on a photodetector. For example, a one dimensional bulk analysis (e.g., in which effective optical property values are used rather than scalar or vector fields) may be used to reduce the amount of computation required to characterize spectral intensity incident on a photodetector. In a further example, Lambert-Beer's law, as shown by Eq. 1, may be derived from Eq. 14 by incorporation of suitable assumptions and mathematical manipulations.

For example, in some embodiments, a patient monitoring system may include a photodetector in contact with a subject's fingertip. The patient monitoring system need not include an explicit light source (e.g., LED, laser), or may include an explicit light source which is optionally not activated. Thermal radiation from the subject, radiation from the environment attenuated by the subject, or both, may be detected by the photodetector. Physiological oscillations or other changes of the subject may cause corresponding changes in one or more radiative properties of the subject (e.g., emission, absorption, scattering, transmission), which may change the spectral intensity incident on the photodetector. Physiological information such as, for example, pulse rate, respiration rate, or other physiological parameters may be derived based at least in part on the incident spectral intensity.

In some embodiments, a photodetector may detect radiation attenuated by a patient, but need not compute, model, or otherwise incorporate radiative phenomena quantitatively when determining physiological information. In some embodiments, no radiative properties need be quantitatively computed, tabulated nor used to determine physiological information. For example, the signal from a photodetector may include information relating to radiative properties of a patient (e.g., a change in arterial compliance, a pulse rate). A patient monitoring system may apply any suitable mathematical manipulation such as a wavelet transform to determine respiration rate, without first computing radiative information. Physiological information may be determined based at least in part on a signal from a photodetector by any suitable mathematical manipulation which may, but need not, include quantitative radiative information.

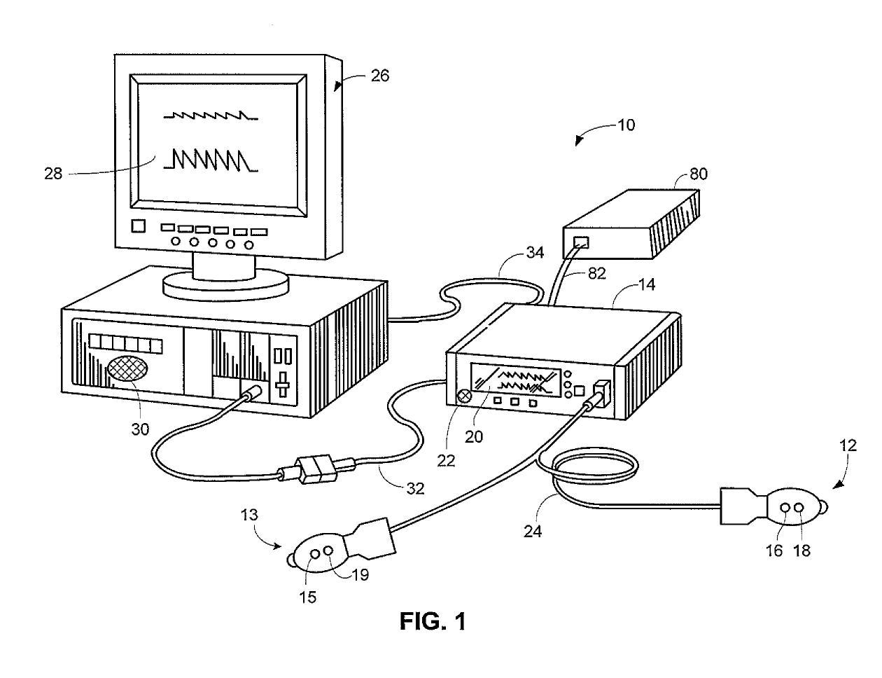

FIG. 1 is a perspective view of an embodiment of a patient monitoring system 10. System 10 may include sensor unit 12 and monitor 14. In an embodiment, sensor unit 12 may be part of a continuous, non-invasive blood pressure (CNIBP) monitoring system and/or an oximeter. Sensor unit 12 may include an emitter 16 for emitting light at one or more wavelengths into a patient's tissue. A detector 18 may also be provided in sensor 12 for detecting the light originally from emitter 16 that emanates from the patient's tissue after passing through the tissue. Any suitable physical configuration of emitter 16 and detector 18 may be used. In some embodiments, sensor unit 12 may include multiple emitters and/or detectors, which may be spaced apart. In some embodiments, sensor 12 need not include an emitter (e.g., emitter 16). In some embodiments, sensor 12 may include emitter 16, but emitter 16 need not be activated (e.g., need not emit light received by a photodetector).

System 10 may also include one or more additional sensor units, such as sensor unit 13, which may take the form of any of the embodiments described herein with reference to sensor unit 12. For example, sensor unit 13 may include emitter 15 and detector 19. Sensor unit 13 may be the same type of sensor unit as sensor unit 12, or sensor unit 13 may be of a different sensor unit type than sensor unit 12. Sensor units 12 and 13 may be capable of being positioned at two different locations on a subject's body; for example, sensor unit 12 may be positioned on a patient's forehead, while sensor unit 13 may be positioned at a patient's fingertip.

Sensor units 12 and 13 may each detect any signal that carries information about a patient's physiological state, such as an electrocardiograph signal, arterial line measurements, or the pulsatile force exerted on the walls of an artery using, for example, oscillometric methods with a piezoelectric transducer. According to another embodiment, system 10 may include a plurality of sensors forming a sensor array in lieu of either or both of sensor units 12 and 13. Each of the sensors of a sensor array may be a complementary metal oxide semiconductor (CMOS) sensor. Alternatively, each sensor of an array may be a charged coupled device (CCD) sensor. In an embodiment, a sensor array may be made up of a combination of CMOS and CCD sensors. The CCD sensor may comprise a photoactive region and a transmission region for receiving and transmitting data whereas the CMOS sensor may be made up of an integrated circuit having an array of pixel sensors. Each pixel may have a photodetector and an active amplifier. It will be understood that any type of sensor, including any type of physiological sensor, may be used in one or more of sensor units 12 and 13 in accordance with the systems and techniques disclosed herein. It is understood that any number of sensors measuring any number of physiological signals may be used to determine physiological information in accordance with the techniques described herein.

In some embodiments, emitter 16 and detector 18 may be on opposite sides of a digit such as a finger or toe, in which case the light that is emanating from the tissue has passed completely through the digit. In some embodiments, emitter 16 and detector 18 may be arranged so that light from emitter 16 penetrates the tissue and is reflected by the tissue into detector 18, such as in a sensor designed to obtain pulse oximetry data from a patient's forehead. In some embodiments, sensor 12 need not include emitter 16, but may include detector 18 which may be arranged to detect electromagnetic radiation attenuated from a patient.

In some embodiments, sensor unit 12 may be connected to and draw its power from monitor 14 as shown. Sensor unit 12 may draw relatively less power when not activated than when activated. In another embodiment, the sensor may be wirelessly connected to monitor 14 and include its own battery or similar power supply (not shown). Monitor 14 may be configured to calculate physiological parameters (e.g., pulse rate, respiration rate, blood pressure, blood oxygen saturation) based at least in part on data relating to light emission, detection, or both, received from one or more sensor units such as sensor units 12 and 13. In some embodiments, the calculations may be performed on the sensor units or an intermediate device and the result of the calculations may be passed to monitor 14. Further, monitor 14 may include a display 20 configured to display the physiological parameters or other information about the system. In the embodiment shown, monitor 14 may also include a speaker 22 to provide an audible sound that may be used in various other embodiments, such as for example, sounding an audible alarm in the event that a patient's physiological parameters are not within a predefined normal range. In some embodiments, the monitor 14 includes a blood pressure monitor. In some embodiments, the system 10 includes a stand-alone blood pressure monitor in communication with the monitor 14 via a cable or a wireless network link.

In some embodiments, sensor unit 12 may be communicatively coupled to monitor 14 via a cable 24. However, in other embodiments, a wireless transmission device (not shown) or the like may be used instead of or in addition to cable 24. For example, in some embodiments, sensor unit 12, monitor 14, or both, may include a transceiver, transponder, transmitter, receiver, antenna, any other suitable component for providing wireless communication, or any combination thereof.

In the illustrated embodiment, system 10 includes a multi-parameter patient monitor 26. The monitor 26 may include a cathode ray tube display, a liquid crystal display (LCD), light emitting diode (LED) display (e.g., organic LED display), projection display, a plasma display, or any other type suitable type of display, or any combination thereof. Multi-parameter patient monitor 26 may be configured to calculate physiological parameters and to provide a display 28 for information from monitor 14 and from other medical monitoring devices or systems (not shown). For example, multi-parameter patient monitor 26 may be configured to display an estimate of a patient's blood oxygen saturation generated by monitor 14 (referred to as an “SpO₂” measurement), pulse rate information from monitor 14 and blood pressure from monitor 14 on display 28. Multi-parameter patient monitor 26 may include a speaker 30. In some embodiments, multi-parameter patient monitor 26 may be configured to display an estimate of a patient's respiration rate generated by monitor 14.

Monitor 14 may be communicatively coupled to multi-parameter patient monitor 26 via a cable 32 or 34 that is coupled to a sensor input port or a digital communications port, respectively and/or may communicate wirelessly (not shown). In some embodiments, monitor 14 and/or multi-parameter patient monitor 26 may be coupled to a network to enable the sharing of information with servers or other workstations (not shown). Monitor 14 may be powered by a battery (not shown), a conventional power source such as a wall outlet, a photovoltaic cell, any other suitable power source, or any combination thereof.

Calibration device 80, which may be powered by monitor 14 via cable 82, a battery, a conventional power source such as a wall outlet, or any other suitable power source, may include any suitable signal calibration device. Calibration device 80 may be communicatively coupled to monitor 14 via cable 82, and/or may communicate wirelessly (not shown). In some embodiments, calibration device 80 may be completely integrated within monitor 14. For example, calibration device 80 may take the form of any invasive or non-invasive blood pressure monitoring or measuring system used to generate reference blood pressure measurements for use in calibrating a CNIBP monitoring technique as described herein. Calibration device 80 may include, for example, an aneroid or mercury sphygmomanometer and occluding cuff, a pressure sensor inserted directly into a suitable artery of a patient, an oscillometric device or any other device or mechanism used to sense, measure, determine, or derive a reference physiological measurement (e.g., blood pressure, pulse rate). In some embodiments, calibration device 80 may include a manual input device (not shown) used by an operator to manually input reference signal measurements obtained from some other source (e.g., an external invasive or non-invasive physiological measurement system).

In some embodiments, calibration device 80 may include a respirometer, stethoscope, any other suitable reference respiratory information (e.g., respiration rate, respiration abnormality). In some embodiments, calibration device 80 may include a manual input device (not shown) used by an operator to manually input reference signal measurements obtained from some other source (e.g., manually counting a patient's breaths per minute).

Calibration device 80 may also access reference signal measurements stored in memory (e.g., RAM, ROM, or a storage device). For example, in some embodiments, calibration device 80 may access reference blood pressure measurements from a relational database stored within calibration device 80, monitor 14, or multi-parameter patient monitor 26. The reference blood pressure measurements generated or accessed by calibration device 80 may be updated in real-time, resulting in a continuous source of reference blood pressure measurements for use in continuous or periodic calibration. Alternatively, reference blood pressure measurements generated or accessed by calibration device 80 may be updated periodically, and calibration may be performed on the same periodic cycle or a different periodic cycle. Reference blood pressure measurements may be generated when recalibration is triggered.

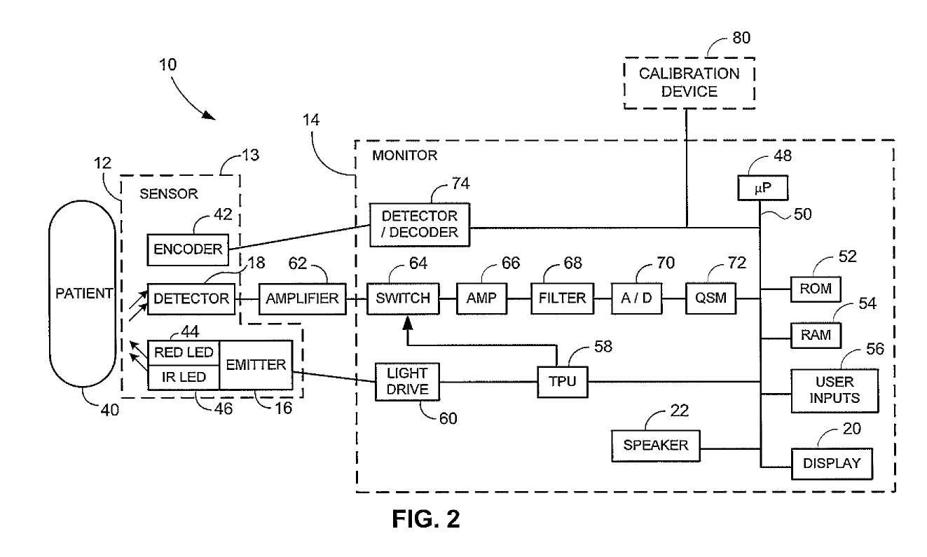

FIG. 2 is a block diagram of a patient monitoring system, such as patient monitoring system 10 of FIG. 1, which may be coupled to a patient 40 in accordance with an embodiment. Certain illustrative components of sensor unit 12 and monitor 14 are illustrated in FIG. 2. Because sensor units 12 and 13 may include similar components and functionality, only sensor unit 12 will be discussed in detail for ease of illustration. It will be understood that any of the concepts, components, and operation discussed in connection with sensor unit 12 may be applied to sensor unit 13 as well (e.g., emitter 16 and detector 18 of sensor unit 12 may be similar to emitter 15 and detector 19 of sensor unit 13). It will be noted that patient monitoring system 10 may include one or more additional sensor units or probes, which may take the form of any of the embodiments described herein with reference to sensor units 12 and 13 (FIG. 1). These additional sensor units included in system 10 may take the same form as sensor unit 12, or may take a different form. In an embodiment, multiple sensors (distributed in one or more sensor units) may be located at multiple different body sites on a patient.

Sensor unit 12 may include emitter 16, detector 18, and encoder 42. In the embodiment shown, emitter 16 may be configured to emit at least two wavelengths of light (e.g., Red and IR) into a patient's tissue 40. Hence, emitter 16 may include a Red light emitting explicit light source such as Red light emitting diode (LED) 44 and an IR light emitting explicit light source such as IR LED 46 for emitting light into the patient's tissue 40 at the wavelengths used to calculate the patient's physiological parameters. In one embodiment, the Red wavelength may be between about 600 nm and about 700 nm, and the IR wavelength may be between about 800 nm and about 1000 nm. In embodiments where a sensor array is used in place of single sensor, each sensor may be configured to emit a single wavelength. For example, a first sensor emits only a Red light while a second emits only an IR light. In another example, the wavelengths of light used are selected based on the specific location of the sensor.

It will be understood that, as used herein, the term “light” may refer to energy produced by radiation sources and may include one or more of ultrasound, radio, microwave, millimeter wave, infrared, visible, ultraviolet, gamma ray or X-ray electromagnetic radiation. As used herein, light may also include any wavelength within the radio, microwave, infrared, visible, ultraviolet, or X-ray spectra, and that any suitable wavelength of electromagnetic radiation may be appropriate for use with the present techniques. Detector 18 may be chosen to be specifically sensitive to the chosen targeted energy spectrum of the emitter 16.

In an embodiment, detector 18 may be configured to detect the intensity of light at the Red and IR wavelengths. Alternatively, each sensor in the array may be configured to detect an intensity of a single wavelength. In operation, light may enter detector 18 after passing through the patient's tissue 40. Detector 18 may convert the intensity of the received light into an electrical signal. The light intensity is directly related to the absorbance and/or reflectance of light in the tissue 40. That is, when more light at a certain wavelength is absorbed or reflected, less light of that wavelength is received from the tissue by the detector 18. After converting the received light to an electrical signal, detector 18 may send the signal to monitor 14, where physiological parameters may be calculated based on the absorption of the Red and IR wavelengths in the patient's tissue 40.

In an embodiment, encoder 42 may contain information about sensor 12, such as what type of sensor it is (e.g., whether the sensor is intended for placement on a forehead or digit) and the wavelengths of light emitted by emitter 16. This information may be used by monitor 14 to select appropriate algorithms, lookup tables and/or calibration coefficients stored in monitor 14 for calculating the patient's physiological parameters.

Encoder 42 may contain information specific to patient 40, such as, for example, the patient's age, weight, and diagnosis. This information about a patient's characteristics may allow monitor 14 to determine, for example, patient-specific threshold ranges in which the patient's physiological parameter measurements should fall and to enable or disable additional physiological parameter algorithms. This information may also be used to select and provide coefficients for equations from which, for example, blood pressure and other measurements may be determined based at least in part on the signal or signals received at sensor unit 12. For example, some pulse oximetry sensors rely on equations to relate an area under a pulse of a photoplethysmograph (PPG) signal to determine blood pressure. These equations may contain coefficients that depend upon a patient's physiological characteristics as stored in encoder 42. Encoder 42 may, for instance, be a coded resistor which stores values corresponding to the type of sensor unit 12 or the type of each sensor in the sensor array, the wavelengths of light emitted by emitter 16 on each sensor of the sensor array, and/or the patient's characteristics. In some embodiments, encoder 42 may include a memory on which one or more of the following information may be stored for communication to monitor 14: the type of the sensor unit 12; the wavelengths of light emitted by emitter 16; the particular wavelength each sensor in the sensor array is monitoring; a signal threshold for each sensor in the sensor array; any other suitable information; or any combination thereof.

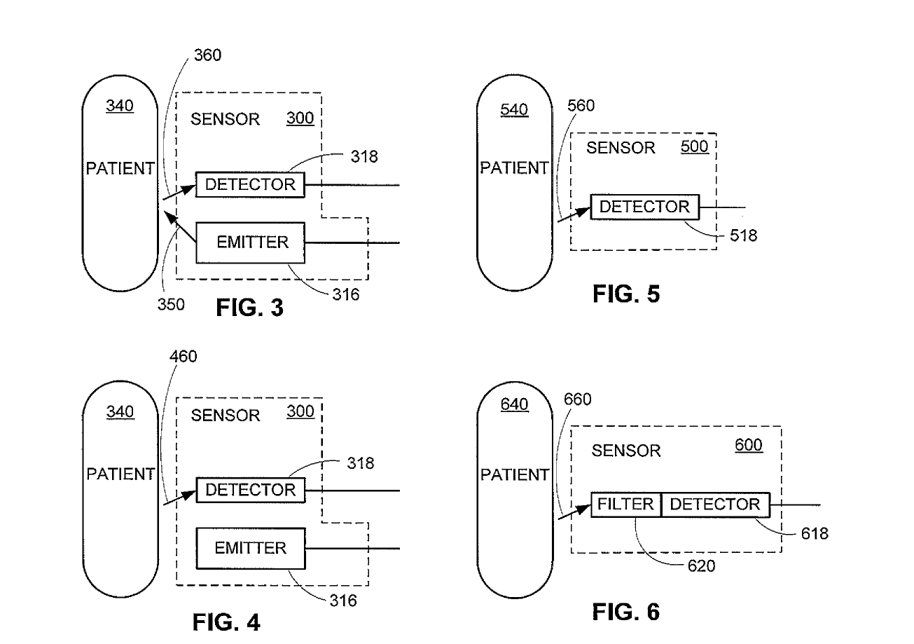

Shown in FIGS. 3-6 are illustrative sensors which may be used to detect photonic signals.

Shown in FIG. 3 is a block diagram of illustrative sensor 300 with an activated explicit light source (i.e., emitter 316) in accordance with an embodiment. Emitter 316 may generate photonic signal 350 at one or more wavelengths. Emitter 316 may be positioned to optically couple to detector 318 by radiative transmission through patient 340 or portion of patient 340 thereof, radiative reflection from patient 340 or surface of patient 340 thereof, or both. Detector 318 may receive incident active photonic signal 360. In some embodiments, sensor 300 may be used to detect an active photonic signal, and output a PPG signal to a patient monitor.

Shown in FIG. 4 is a block diagram of the illustrative sensor 300 of FIG. 3, in which emitter 316 is unactivated, in accordance with some embodiments. In this and other embodiments, emitter 316 of sensor 300 may be turned off, have its photonic output blocked from being attenuated by patient 340, or otherwise be unactivated. Detector 318 may receive incident passive photonic signal 460, which does not originate from an emitter (e.g., emitter 316). In some embodiments, sensor 300 may be used to detect a passive photonic signal and output a PPG signal to a patient monitor. Incident passive photonic signal 460 may have different spectral character (e.g., relative intensity at each wavelength) and/or directional character (e.g., relatively more diffuse, or less directionally pronounced) than incident active photonic signal 360.

Shown in FIG. 5 is a block diagram of illustrative sensor 500 without an explicit light source (e.g., an emitter) in accordance with some embodiments. In some embodiments, detector 518 may detect incident radiation 560 which may include radiative emission from patient 540, radiative reflection from a surface of patient 540, radiative transmission through patient 540 or portion of patient 540 thereof, or any combination thereof. In some embodiments, ambient radiation (e.g., diffuse light) may be attenuated (e.g., absorbed, scattered, reflected) by a patient, and the attenuated ambient radiation may be received by a photodetector of a patient monitoring system. In some embodiments, sensor 500 may be used to detect a passive photonic signal, and output a PPG signal to a patient monitor.

Shown in FIG. 6 is a block diagram of illustrative sensor 600 with optical filter 620 in place in accordance with some embodiments. Optical filter 620 may be any suitable device for filtering optical radiation such, for example, a neutral density filter, spectral filter (e.g., band pass filter), directional filter (e.g., blinds), any other suitable type of optical filter, or any combination thereof. Optical filter 620 may reduce the intensity of some or all wavelengths of incident radiation 660 on detector 618. In some embodiments, sensor 600 may be used to detect a photonic signal, and output a PPG signal to a patient monitor.

In some embodiments, one or more optical filters (e.g., optical filter 620), lenses, mirrors, shutters, apertures, optical choppers, windows, optical fibers, any other suitable optical components, or any combination thereof may be included in any of sensors 300, 400, 500, and 600 of FIGS. 3-6, respectively.

In some embodiments, more than one photodetector, such as an array of photodetectors (an “array”), may be used to receive photonic signals. In some embodiments, one or more filters may be applied to a subset photodetectors of a collection photodetectors. For example, a particular optical spectral filter may allow a subset of incident electromagnetic radiation to pass to the subset of photodetectors. In some embodiments, more than one optical spectral filter may be applied to more than one subset of photodetectors of an array. For example, one subset of photodetectors may be filtered to detect red wavelength radiation, while a second subset may be filtered to simultaneously detect infrared wavelength radiation. In some embodiments, detection of red and infrared wavelength radiation attenuated by a patient may allow for SpO₂ of the patient to be determined.

In some embodiments, more than one photodetector, such as an array may be used to provide an ensemble average of a received photonic signal. For example, the output of unfiltered or similarly filtered photodetectors of an array may be ensemble averaged, time averaged, or otherwise statistically manipulated to provide averaged values. In some embodiments, averaged values may provide increased signal to noise ratios, decreased fluctuations, decreased noise, or other desired changes in parameters relative to non-averaged values.

In some embodiments, signals from detector 18 and encoder 42 may be transmitted to monitor 14. In the embodiment shown, monitor 14 may include a general-purpose microprocessor 48 connected to an internal bus 50. Microprocessor 48 may be adapted to execute software, which may include an operating system and one or more applications, as part of performing the functions described herein. Also connected to bus 50 may be a read-only memory (ROM) 52, a random access memory (RAM) 54, user inputs 56, display 20, and speaker 22.

RAM 54 and ROM 52 are illustrated by way of example, and not limitation. Any suitable computer-readable media may be used in the system for data storage. Computer-readable media are capable of storing information that can be interpreted by microprocessor 48. This information may be data or may take the form of computer-executable instructions, such as software applications, that cause the microprocessor to perform certain functions and/or computer-implemented methods. Depending on the embodiment, such computer-readable media may include computer storage media and communication media. Computer storage media may include volatile and non-volatile, removable and non-removable media implemented in any method or technology for storage of information such as computer-readable instructions, data structures, program modules or other data. Computer storage media may include, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by components of the system.

In the illustrative embodiment shown, a time processing unit (TPU) 58 may provide timing control signals to light drive circuitry 60, which may control when emitter 16 is illuminated and multiplexed timing for Red LED 44 and IR LED 46. TPU 58 may also control the gating-in of signals from detector 18 through amplifier 62 and switching circuit 64. These signals are sampled at the proper time, depending upon which explicit light source is illuminated. The received signal from detector 18 may be passed through amplifier 66, low pass filter 68, and analog-to-digital converter 70. The digital data may then be stored in a queued serial module (QSM) 72 (or buffer) for later downloading to RAM 54 as QSM 72 fills up. In one embodiment, there may be multiple separate parallel paths having components equivalent to amplifier 66, filter 68, and/or A/D converter 70 for multiple light wavelengths or spectra received.

In some embodiments, microprocessor 48 may determine the patient's physiological parameters, such as SpO₂, pulse rate, respiration rate, and/or blood pressure, using various algorithms and/or look-up tables based on the value of the received signals and/or data corresponding to the light received by detector 18. Signals corresponding to information about patient 40, and particularly about the intensity of light emanating from a patient's tissue over time, may be transmitted from encoder 42 to decoder 74. These signals may include, for example, encoded information relating to patient characteristics. Decoder 74 may translate these signals to enable the microprocessor to determine the thresholds based at least in part on algorithms or look-up tables stored in ROM 52. User inputs 56 may be used to enter information about the patient, such as age, weight, height, diagnosis, medications, treatments, and so forth. In an embodiment, display 20 may exhibit a list of values which may generally apply to the patient, such as, for example, age ranges or medication families, which the user may select using user inputs 56.

Optical signals transmitted through tissue may be altered by noise, among other sources. One source of noise of active photonic signals is ambient light that reaches the light detector. Another source of noise is electromagnetic coupling from other electronic instruments. Movement of the patient also introduces noise and affects the signal. For example, the contact between the detector and the skin, or the emitter and the skin, can be temporarily disrupted when movement causes either to move away from the skin. In addition, because blood is a fluid, it responds differently than the surrounding tissue to inertial effects, thus resulting in momentary changes in volume at the point to which the oximeter probe is attached.

Noise (e.g., from patient movement) can degrade a sensor signal relied upon by a care provider, without the care provider's awareness. This is especially true if the monitoring of the patient is remote, the motion is too small to be observed, or the care provider is watching the instrument or other parts of the patient, and not the sensor site. Processing sensor signals (e.g., PPG signals) may involve operations that reduce the amount of noise present in the signals or otherwise identify noise components in order to prevent them from affecting measurements of physiological parameters derived from the sensor signals.

Pulse oximeters, in addition to providing other information, can be utilized for continuous non-invasive blood pressure monitoring. As described in Chen et al., U.S. Pat. No. 6,599,251, the entirety of which is incorporated herein by reference, PPG and other pulse signals obtained from multiple probes can be processed to calculate the blood pressure of a patient. In particular, blood pressure measurements may be derived based on a comparison of time differences between certain components of the pulse signals detected at each of the respective probes. As described in U.S. patent application Ser. No. 12/242,238, filed on Sep. 30, 2008 and entitled “Systems and Methods For Non-Invasive Blood Pressure Monitoring,” the entirety of which is incorporated herein by reference, blood pressure can also be derived by processing time delays detected within a single PPG or pulse signal obtained from a single pulse oximeter probe. In addition, as described in U.S. patent application Ser. No. 12/242,867, filed on Sep. 30, 2008 and entitled “Systems and Methods For Non-Invasive Continuous Blood Pressure Determination,” the entirety of which is incorporated herein by reference, blood pressure may also be obtained by calculating the area under certain portions of a pulse signal. Finally, as described in U.S. patent application Ser. No. 12/242,862, filed on Sep. 30, 2008 and entitled “Systems and Methods For Maintaining Blood Pressure Monitor Calibration,” the entirety of which is incorporated herein by reference, a blood pressure monitoring device may be recalibrated in response to arterial compliance changes.

In some embodiments, some CNIBP monitoring techniques utilize two probes or sensors positioned at two different locations on a subject's body. The elapsed time, T, between the arrivals of corresponding points of a pulse signal at the two locations may then be determined using signals obtained by the two probes or sensors. The estimated blood pressure, p, may then be related to the elapsed time, T, by

p=a+b·ln(T) (19)

where a and b are constants that may be dependent upon the nature of the subject and the nature of the signal detecting devices. Other suitable equations using an elapsed time between corresponding points of a pulse signal may also be used to derive an estimated blood pressure measurement.

In some embodiments, Eq. 19 may include a non-linear function which is monotonically decreasing and concave upward in T in a manner specified by the constant parameters (in addition to or instead of the expression of Eq. 19). Eq. 19 may be used to calculate an estimated blood pressure from the time difference T between corresponding points of a pulse signal received by two sensors or probes attached to two different locations of a subject.

In some embodiments, constants a and b in Eq. 19 above may be determined by performing a calibration. The calibration may involve taking a reference blood pressure reading to obtain a reference blood pressure P₀, measuring the elapsed time T₀ corresponding to the reference blood pressure, and then determining values for both of the constants a and b from the reference blood pressure and elapsed time measurement. Calibration may be performed at any suitable time (e.g., once initially after monitoring begins) or on any suitable schedule (e.g., a periodic or event-driven schedule).

In some embodiments, the calibration may include performing calculations mathematically equivalent to

$$a = {c}_{1} + \frac{{c}_{2} ( {P}_{0} - {c}_{1} )}{ln ( {T}_{0} ) + {c}_{2}} \qquad ( 20 )$$

and

$$b = \frac{{P}_{0} - {c}_{1}}{ln ( {T}_{0} ) + {c}_{2}} \qquad ( 21 )$$

to obtain values for the constants a and b, where c₁ and c₂ are parameters that may be determined, for example, based on empirical data.

In an embodiment, the calibration may include performing calculations mathematically equivalent to

a=P₀−(c₃T₀+c₄)ln(T₀) (22)

and

b=c₃T₀+c₄ (23)

where a and b are first and second parameters and c₃ and c₄ are parameters that may be determined, for example, based on empirical data.

Parameters c₁, c₂, c₃, and c₄ may be predetermined constants empirically derived using experimental data from a number of different patients. A single reference blood pressure reading from a patient, including reference blood pressure P₀ and elapsed time T₀ from one or more signals corresponding to that reference blood pressure, may be combined with such inter-patient data to calculate the blood pressure of a patient. The values of P₀ and T₀ may be referred to herein as a calibration point. According to this example, a single calibration point may be used with the predetermined constant parameters to determine values of constants a and b for the patient (e.g., using Eqs. 20 and 21 or 22 and 23). The patient's blood pressure may then be calculated using Eq. 19. Recalibration may be performed by collecting a new calibration point and recalculating the constants a and b used in Eq. 19. Calibration and recalibration may be performed using calibration device 80 (FIG. 1).

In some embodiments, multiple calibration points from a patient may be used to determine the relationship between the patient's blood pressure and one or more PPG signals. This relationship may be linear or non-linear and may be extrapolated and/or interpolated to define the relationship over the range of the collected recalibration data. For example, the multiple calibration points may be used to determine values for parameters c₁ and c₂ or c₃ and c₄ (described above). These determined values will be based on information about the patient (intra-patient data) instead of information that came from multiple patients (inter-patient data). As another example, the multiple calibration points may be used to determine values for parameters a and b (described above). Instead of calculating values of parameters a and b using a single calibration point and predetermined constants, values for parameters a and b may be empirically derived from the values of the multiple calibration points. As yet another example, the multiple calibration points may be used directly to determine the relationship between blood pressure and PPG signals. Instead of using a predefined relationship (e.g., the relationship defined by Eq. 19), a relationship may be directly determined from the calibration points.

Additional examples of continuous and non-invasive blood pressure monitoring techniques are described in Chen et al., U.S. Pat. No. 6,566,251, which is hereby incorporated by reference herein in its entirety. The technique described by Chen et al. may use two sensors (e.g., ultrasound or photoelectric pulse wave sensors) positioned at any two locations on a subject's body where pulse signals are readily detected. For example, sensors may be positioned on an earlobe and a finger, an earlobe and a toe, or a finger and a toe of a patient's body.

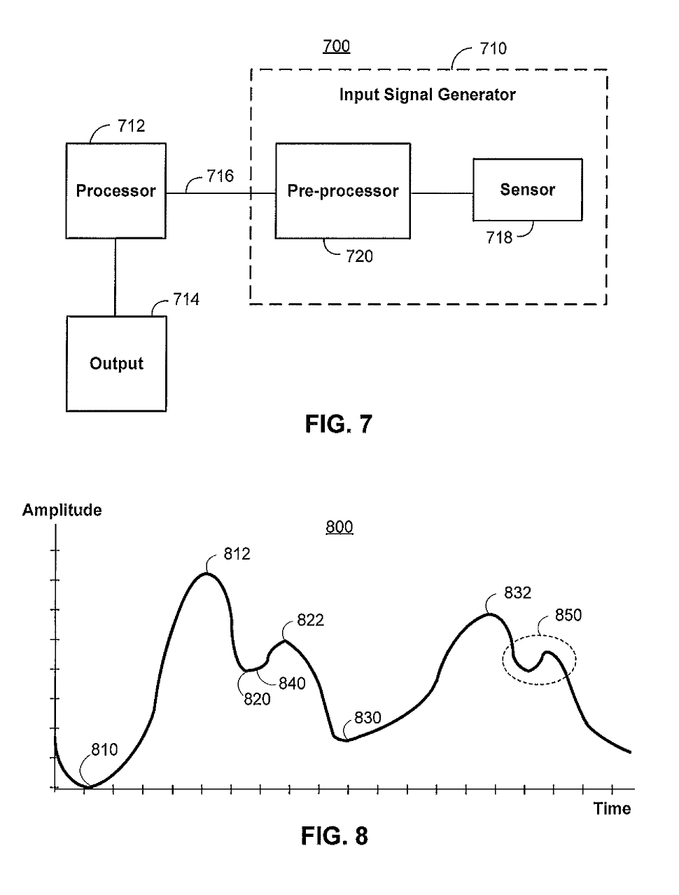

FIG. 7 is an illustrative signal processing system 700 in accordance with an embodiment that may implement the non-invasive blood pressure techniques described herein. In this embodiment, input signal generator 710 generates an input signal 716. As illustrated, input signal generator 710 may include pre-processor 720 coupled to sensor 718, which may provide input signal 716. In an embodiment, pre-processor 720 may be an oximeter and input signal 716 may be a PPG signal. In an embodiment, pre-processor 720 may be any suitable signal processing device and input signal 716 may include one or more PPG signals and one or more other physiological signals, such as an electrocardiogram (ECG) signal. It will be understood that input signal generator 710 may include any suitable signal source, signal generating data, signal generating equipment, or any combination thereof to produce signal 716. Signal 716 may be a single signal, or may be multiple signals transmitted over a single pathway or multiple pathways.

Pre-processor 720 may apply one or more signal processing operations to the signal generated by sensor 718. For example, pre-processor 720 may apply a pre-determined set of processing operations to the signal provided by sensor 718 to produce input signal 716 that can be appropriately interpreted by processor 712, such as performing A/D conversion. Pre-processor 720 may also perform any of the following operations on the signal provided by sensor 718: reshaping the signal for transmission, multiplexing the signal, modulating the signal onto carrier signals, compressing the signal, encoding the signal, and filtering the signal.

In some embodiments, signal 716 may include PPG signals at one or more frequencies, such as a Red PPG signal and an IR PPG signal. In an embodiment, signal 716 may include signals measured at one or more sites on a patient's body, for example, a patient's finger, toe, ear, arm, or any other body site. In some embodiments, signal 716 may include multiple types of signals (e.g., one or more of an ECG signal, an EEG signal, an acoustic signal, an optical signal, a signal representing a blood pressure, and a signal representing a pulse rate). Signal 716 may be any suitable biosignal or signals, such as, for example, electrocardiogram, electroencephalogram, electrogastrogram, electromyogram, pulse rate signals, pathological sounds, ultrasound, or any other suitable biosignal.

In some embodiments, signal 716 may be coupled to processor 712. Processor 712 may be any suitable software, firmware, hardware, or combination thereof for processing signal 716. For example, processor 712 may include one or more hardware processors (e.g., integrated circuits), one or more software modules, computer-readable media such as memory, firmware, or any combination thereof. Processor 712 may, for example, be a computer or may be one or more chips (i.e., integrated circuits). Processor 712 may, for example, be configured of analog electronic components. Processor 712 may perform the calculations associated with the information determination techniques of the present disclosure as well as the calculations associated with any calibration of processing system 700 or other auxiliary functions. For example, processor 712 may locate one or more fiducial points in one or more signals, determine one or more DPTTs, and compute one or more of a systolic blood pressure, a diastolic blood pressure and a mean arterial pressure. Processor 712 may perform any suitable signal processing of signal 716 to filter signal 716, such as any suitable band-pass filtering, adaptive filtering, closed-loop filtering, any other suitable filtering, and/or any combination thereof. Processor 712 may also receive input signals from additional sources (not shown). For example, processor 712 may receive an input signal containing information about treatments provided to the patient. Additional input signals may be used by processor 712 in any of the calculations or operations it performs in accordance with processing system 700.

Processor 712 may be coupled to one or more memory devices (not shown) or incorporate one or more memory devices such as any suitable volatile memory device (e.g., RAM, registers, etc.), non-volatile memory device (e.g., ROM, EPROM, magnetic storage device, optical storage device, flash memory, etc.), or both. The memory may be used by processor 712 to, for example, store data corresponding to blood pressure monitoring, including current blood pressure calibration values, blood pressure monitoring calibration thresholds, and patient blood pressure history. In some embodiments, processor 712 may store physiological measurements or previously received data from signal 716 in a memory device for later retrieval. In some embodiments, processor 712 may store calculated values, such as a systolic blood pressure, a diastolic blood pressure, a blood oxygen saturation, a differential pulse transit time, a fiducial point location or characteristic, or any other calculated values, in a memory device for later retrieval.

Processor 712 may be coupled to a calibration device. This coupling may take any of the forms described above with reference to calibration device 80 within system 10. For example, the calibration device may be a stand-alone device that may be in wireless communication with processor 712, or may be completely integrated with processor 712.

Processor 712 may be coupled to a calibration device that may generate, or receive as input, reference measurements for use in calibration calculations. This coupling may occur through a recalibration signal transmitted via a wired or wireless communications path. In an embodiment, processor 712 is capable of transmitting a command to calibration device 80 to initiate a recalibration procedure.

Processor 712 may be coupled to output 714. Output 714 may be any suitable output device such as one or more medical devices (e.g., a medical monitor that displays various physiological parameters, a medical alarm, or any other suitable medical device that either displays physiological parameters or uses the output of processor 712 as an input), one or more display devices (e.g., monitor, PDA, mobile phone, any other suitable display device, or any combination thereof), one or more audio devices, one or more memory devices (e.g., hard disk drive, flash memory, RAM, optical disk, any other suitable memory device, or any combination thereof), one or more printing devices, any other suitable output device, or any combination thereof.

It will be understood that system 700 may be incorporated into system 10 (FIGS. 1 and 2) in which, for example, input signal generator 710 may be implemented as parts of sensor units 12 and 13 (FIGS. 1 and 2) and monitor 14 (FIGS. 1 and 2) and processor 712 may be implemented as part of monitor 14 (FIGS. 1 and 2). In some embodiments, portions of system 700 may be configured to be portable. For example, all or part of system 700 may be embedded in a small, compact object carried with or attached to the patient (e.g., a watch, other piece of jewelry, or a cellular telephone). In such embodiments, a wireless transceiver (not shown) may also be included in system 700 to enable wireless communication with other components of system 10 (FIGS. 1 and 2). As such, system 10 (FIGS. 1 and 2) may be part of a fully portable and continuous patient monitoring solution. In some embodiments, a wireless transceiver (not shown) may also be included in system 700 to enable wireless communication with other components of system 10. For example, pre-processor 720 may output signal 716 over BLUETOOTH, 802.11, WiFi, WiMax, cable, satellite, Infrared, or any other suitable transmission scheme. In some embodiments, a wireless transmission scheme may be used between any communicating components of system 700.

Pre-processor 720 or processor 712 may determine the locations of pulses within a periodic signal 716 (e.g., a PPG signal) using a pulse detection technique. For ease of illustration, the following pulse detection techniques will be described as performed by processor 712, but any suitable processing device (e.g., pre-processor 720) may be used to implement any of the techniques described herein.

An illustrative PPG signal 800 is depicted in FIG. 8. Processor 712 may receive PPG signal 800, and may identify local minimum point 810, local maximum point 812, local minimum point 820, and local maximum point 822 in the PPG signal 800. Processor 712 may pair each local minimum point with an adjacent maximum point. For example, processor 712 may pair points 810 and 812 to identify one segment, points 812 and 820 to identify a second segment, points 820 and 822 to identify a third segment and points 822 and 830 to identify a fourth segment. The slope of each segment may be measured to determine whether the segment corresponds to an upstroke portion of the pulse (e.g., a positive slope) or a downstroke portion of the pulse (e.g., a negative slope) portion of the pulse. A pulse may be defined as a combination of at least one upstroke and one downstroke. For example, the segment identified by points 810 and 812 and the segment identified by points 812 and 820 may define a pulse.

According to an embodiment, PPG signal 800 may include a dichrotic notch 850 or other notches (not shown) in different sections of the pulse (e.g., at the beginning (referred to as an ankle notch), in the middle (referred to as a dichrotic notch), or near the top (referred to as a shoulder notch)). Processor 712 may identify notches and either utilize or ignore them when detecting the pulse locations. In some embodiments, processor 712 may compute the second derivative of the PPG signal to find the local minima and maxima points and may use this information to determine a location of, for example, a dichrotic notch. Additionally, processor 712 may interpolate between points in signal 716 or between points in a processed signal using any interpolation technique (e.g., zero-order hold, linear interpolation, and/or higher-order interpolation techniques). Some pulse detection techniques that may be performed by processor 712 are described in more detail in co-pending, commonly assigned U.S. patent application Ser. No. 12/242,908, filed Sep. 30, 2008 and entitled “SYSTEMS AND METHODS FOR DETECTING PULSES IN A PPG SIGNAL,” which is incorporated by reference herein in its entirety.

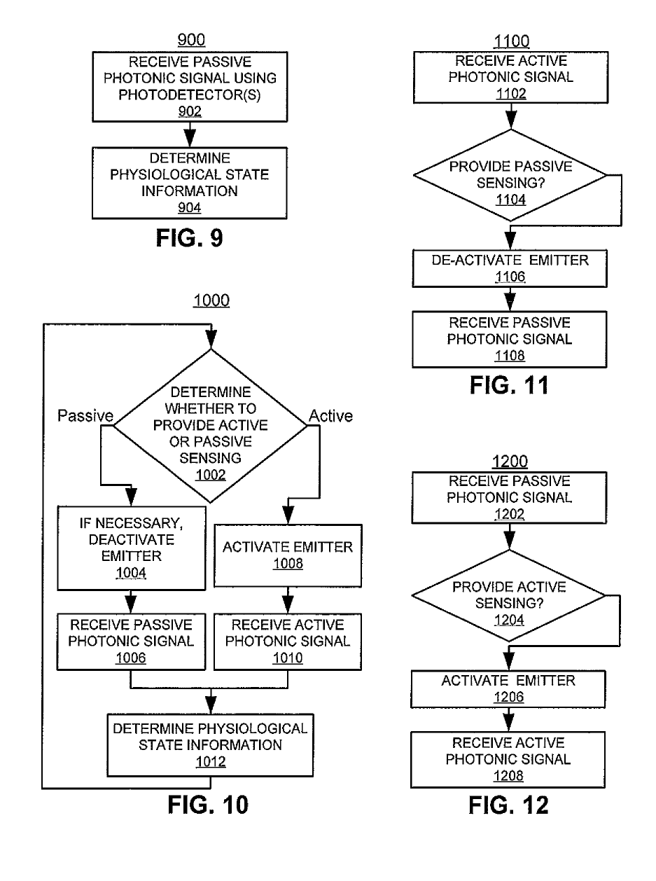

Shown in FIG. 9 is flow diagram 900 of illustrative steps for determining physiological information in accordance with an embodiment.

Step 902 may include a receiving any suitable type of passive photonic signal. Step 902 may include using any suitable type of photodetector such as, for example, a photodiode, a photomultiplier tube, a CCD, any other suitable photodetector, or any combination or array thereof to receive a photonic signal. In some embodiments, the passive photonic signal of step 902 may be generated without the use of an explicit light source (e.g., an emitter). In some embodiments, the passive photonic signal of step 902 may be generated with an explicit light source included as a part of an optical system, but the explicit light source need not be activated.

In some embodiments, step 902 may include applying signal processing techniques to a signal received from a photodetector (e.g., an electrical signal). For example, step 902 may include applying sampling, filtering (e.g., using a bandpass filter), amplifying, inverting, modulating, demodulating, multiplexing, any other suitable signal processing technique or any combination thereof to a signal received from a photodetector. For example, a patient monitoring system may be configured to receive, process, or both, more than one passive photoplethysmograph signal from one or more photodetectors.

Step 904 may include determining physiological information associated with a patient based at least in part on a passive photonic signal derived at least in part from a photodetector. Physiological information may include any suitable physiological parameter, or other parameter, such as, for example, pulse rate, respiration rate, any other parameter, changes in value thereof, or any suitable combination thereof.

Step 904 may include applying any suitable type of mathematical manipulation of a received signal from a photodetector, database searching, or other suitable computational technique. In some embodiments, step 904 may include performing a discrete or continuous transform, a Fourier transform, any other suitable transformation, a statistical operation, a database search, any other suitable computation, or any combination thereof on a signal received from a photodetector. For example, in some embodiments, a patient monitoring system may perform a discrete or continuous wavelet transform on a received passive photoplethysmograph signal from a photodetector optically coupled to a patient. The patient monitoring system may compute a respiration rate, pulse rate, or other physiological information, or any changes thereof, based on the output of the wavelet transform.

Shown in FIG. 10 is flow diagram 1000 of illustrative steps for determining whether to provide active or passive sensing in accordance with an embodiment.

Step 1002 may include a patient monitoring system determining whether to provide active or passive sensing. A patient monitoring system may determine whether to provide active or passive sensing based at least in part on a predetermined schedule, a signal to noise ratio of a received signal, a change in a received signal, power consumption, a user input, a value of a physiological parameter, any other suitable information, or any combination thereof. For example, patient monitoring system may determine whether to provide active or passive sensing at a regular time interval. In a further example, a patient monitoring system may determine whether to provide active or passive sensing in response to the signal to noise ratio of a photoplethysmograph signal falling below or increasing above a threshold. In a further example, a patient monitoring system may determine that passive sensing is to be provided to reduce power consumption. Any suitable criteria may be used to schedule the determination of step 1002, perform the determination of step 1002, or both.

If it is determined at step 1002 to provide passive sensing, a patient monitoring system need not activate, or may deactivate, an explicit light source (e.g., an emitter) at step 1004. In some embodiments, step 1004 may include switching off a power supply to an explicit light source, closing a shutter, redirecting the output of the light source, any other suitable technique for providing a passive photoplethysmograph signal from a photodetector, or any suitable combination thereof. In some embodiments, for example, no explicit light source may be included, and step 1004 need not be performed. Step 1004, step 1002, or both, may include a determination as to whether it may be necessary to deactivate an explicit light source to provide passive sensing.

Step 1006 may include a patient monitoring system receiving a passive photonic signal at a suitable photodetector. In some embodiments, step 1006 may include applying signal processing techniques to an output signal of the photodetector (e.g., an electrical signal). For example, step 1006 may include applying sampling, filtering (e.g., using a bandpass filter), amplifying, inverting, modulating, demodulating, multiplexing, any other suitable signal processing technique or any combination thereof to a signal received from a photodetector. In some embodiments, a patient monitoring system may be configured to receive, process, or both, more than one passive photoplethysmograph signal from more than one photodetector.

In some embodiments, step 1004 need not be performed in order for a patient monitoring system to provide passive sensing. For example, a patient monitoring system without an explicit light source may provide passive sensing by receiving a signal from a suitable photodetector suitably coupled to a patient, as shown by step 1006.

If it is determined at step 1002 to provide active sensing, a patient monitoring system may activate an explicit light source at step 1008. In some embodiments, step 1008 may include switching on a power supply to an explicit light source, controlling the power delivered to the light source, controlling the intensity of output of the light source, opening a shutter, redirecting the output of the light source, any other suitable technique for providing an active photoplethysmograph signal, or any suitable combination thereof.

In some embodiments, step 1002, step 1008, or both, may include a patient monitoring system determining how much power is to be delivered to an explicit light source. The patient monitoring system may determine the extent to which the explicit light source is to be activated. For example, a patient monitoring system may determine how much power to deliver to an explicit light source based at least in part on a signal to noise ratio of a signal received from a photodetector. In a further example, a patient monitoring system may determine how much power to deliver to an explicit light source based at least in part on a comparison between a signal received from a photodetector, or feature derived thereof, and a threshold.

Step 1010 may include a patient monitoring system receiving an active photonic signal at a suitable photodetector. In some embodiments, step 1010 may include applying signal processing techniques to a signal received from a photodetector (e.g., an electrical signal). For example, step 1010 may include sampling, filtering (e.g., using a bandpass filter), amplifying, inverting, modulating, demodulating, multiplexing, any other suitable signal processing technique or any combination thereof to a signal received from a photodetector. In some embodiments, a patient monitoring system may be configured to receive, process, or both, more than one active photonic signal. For example, a patient monitoring system may be configured to receive two photoplethysmograph signals from two photodetectors optically coupled to a patient to determine DPTT.

In some embodiments, a patient monitoring system may determine that both active and passive sensing are to be provided at the same or different times. Any suitable schedule of active and passive sensing may be used, including simultaneous active and passive sensing. For example, a patient monitoring system may include two photodetectors and two light sources suitably arranged to optically interact with the respective photodetectors. The patient monitoring system may deactivate one light source, and activate the other light source so that at a given time one passive photonic signal is received and one active photonic signal is received.

Step 1012 may include determining physiological information based at least in part on a signal received from a photodetector which may be optically coupled to a patient. The photodetector may receive photonic signal which may be active, passive, or any combination thereof. Physiological information may include any suitable physiological parameter, or other parameter derived at least in part from a patient, such as, for example, pulse rate, respiration rate, blood pressure, SpO₂, changes in value thereof, or any suitable combination thereof.

Step 1012 may include applying any suitable type of mathematical manipulation of a signal received from a photodetector, database searching, or other suitable computational technique. For example, one or more metric values derived at least in part from a signal received from a photodetector may be computed, and the metric values may be compared to stored values in a database. In some embodiments, step 1012 may include performing a transform on the received signal, performing statistical operations on the received signal, performing a database search based at least in part on received signal value, or performing any other suitable computation or combination thereof. For example, in some embodiments, a patient monitoring system may perform a continuous or discrete wavelet transform on a received PPG signal from a photodetector optically coupled to a patient. The patient monitoring system may compute a respiration rate, pulse rate, or other physiological parameter, or any changes thereof, based on the output of the wavelet transform.

In some embodiments, a patient monitoring system may determine physiological information at step 1012 based on a signal received from a photodetector, and then perform step 1002. For example, a patient monitoring system may perform (e.g., repeat) step 1002 based at least in part on the value of a physiological parameter, changes in value of a physiological parameter, one or more threshold values, signal metrics (e.g., signal to noise ratio, signal fluctuations), user input, any other criteria, or any combination thereof.

Shown in FIG. 11 is flow diagram 1100 of illustrative steps for determining whether to provide passive sensing in accordance with an embodiment.

Step 1102 may include a patient monitoring system receiving one or more active photonic signals at a suitable photodetector. The photodetector may transmit a signal (e.g., an electrical signal), based at least in part on the photonic signal, which may be received by a patient monitoring system. In some embodiments, step 1102 may include applying signal processing techniques to one or more signals received from a photodetector. For example, step 1102 may include sampling, filtering (e.g., using a bandpass filter), amplifying, inverting, modulating, demodulating, multiplexing, any other suitable signal processing technique or any combination thereof to a received signal.

Step 1104 may include a patient monitoring system determining whether to provide passive sensing. A patient monitoring system may determine whether to provide passive sensing based at least in part on a predetermined schedule, a signal to noise ratio of a received signal, a change in a received signal, power consumption, a user input, a value of a physiological parameter, any other suitable information, or any combination thereof. For example, patient monitoring system may determine to provide passive sensing at a regular time interval. In a further example, a patient monitoring system may determine to provide passive sensing in response to the signal to noise ratio of a signal received from a photodetector increasing above a threshold. In a further example, a patient monitoring system may determine to provide passive sensing if the power consumption of the patient monitoring system or components thereof are determined to be above a threshold. Any suitable criteria may be used to schedule the determination of step 1104, perform the determination of step 1104, or both.

In some embodiments, step 1106 may include switching off a power supply to an explicit light source, closing a shutter, redirecting the output of the light source, any other suitable technique for providing a passive photonic signal, or any suitable combination thereof.

Step 1108 may include a patient monitoring system receiving a passive photonic signal at a suitable photodetector. In some embodiments, step 1108 may include applying signal processing techniques to a signal received from a photodetector. For example, step 1108 may include applying sampling, filtering (e.g., using a bandpass filter), amplifying, inverting, modulating, demodulating, multiplexing, any other suitable signal processing technique, or any combination thereof to a received signal. In some embodiments, a patient monitoring system may be configured to receive, process, or both, more than one passive photonic signal.

Shown in FIG. 12 is flow diagram 1200 of illustrative steps for determining whether to provide active sensing in accordance with an embodiment.

Step 1202 may include a patient monitoring system receiving a passive photonic signal at a suitable photodetector. In some embodiments, step 1202 may include applying signal processing techniques to a signal received from a photodetector. For example, step 1202 may include sampling, filtering (e.g., using a bandpass filter), amplifying, inverting, modulating, demodulating, multiplexing, any other suitable signal processing technique or any combination thereof to a signal received from a photodetector. In some embodiments, a patient monitoring system may be configured to receive, process, or both, more than one passive photonic signal.

Step 1204 may include a patient monitoring system determining whether to provide active sensing. A patient monitoring system may determine whether to provide active sensing based at least in part on a predetermined schedule, a signal to noise ratio of a received signal, a change in a received signal, power consumption, a user input, a value of a physiological parameter, any other suitable information, or any combination thereof. For example, patient monitoring system may determine to provide active sensing at a regular time interval. In a further example, a patient monitoring system may determine to provide active sensing in response to the signal to noise ratio of a photonic signal falling below a threshold. In a further example, a patient monitoring system may determine to provide active sensing based at least in part on a comparison between a signal received from a photodetector, or feature derived thereof, and a threshold. Any suitable criteria may be used to schedule the determination of step 1204, perform the determination of step 1204, or both.

In some embodiments, step 1204 may include a patient monitoring system determining how much power is to be delivered to an explicit light source (e.g., an emitter). The patient monitoring system may determine the extent to which the explicit light source is to be activated. For example, a patient monitoring system may determine to the extent to which an explicit light source is to be activated based at least in part on a signal to noise ratio of a signal received from a photodetector.

In some embodiments, step 1206 may include switching on a power supply to an explicit light source, controlling the power delivered to the light source, controlling the intensity of the output of the light source, opening a shutter, redirecting the output of the light source, any other suitable technique for providing an active photonic signal, or any suitable combination thereof.

Step 1208 may include a patient monitoring system receiving an active photonic signal at a suitable photodetector. In some embodiments, step 1208 may include applying signal processing techniques to a signal received from a photodetector (e.g., an electrical signal). For example, step 1208 may include applying sampling, filtering (e.g., using a bandpass filter), amplifying, inverting, modulating, demodulating, multiplexing, any other suitable signal processing technique or any combination thereof to a signal received from a photodetector. In some embodiments, a patient monitoring system may be configured to receive, process, or both, more than one signal received from more than one photodetector.

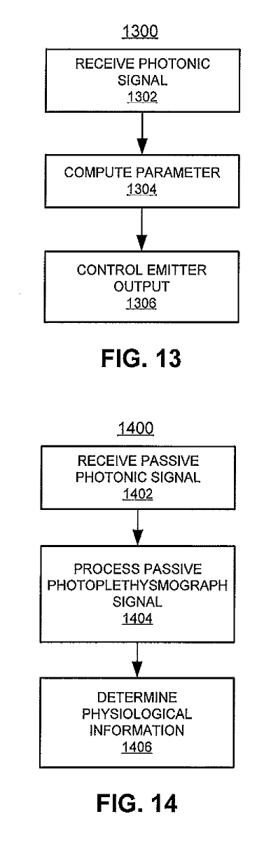

Shown in FIG. 13 is flow diagram 1300 of illustrative steps for determining physiological information in accordance with an embodiment.

Step 1302 may include a patient monitoring system receiving a signal (e.g., an electrical signal) from a photodetector which may receive any suitable type of active or passive photonic signal. Step 1302 may include using any suitable type of photodetector such as, for example, a photodiode, a photomultiplier tube, a CCD, any other suitable photodetectors, or any combination or arrays thereof to receive a photonic signal. In some embodiments, the photonic signal of step 1302 may be generated without an explicit light source included as a part of the monitoring system. In some embodiments, the photonic signal of step 1302 may be generated with an explicit light source included as a part of the monitoring system.

In some embodiments, step 1302 may include applying signal processing techniques to a signal received from a photodetector. For example, step 1302 may include sampling, filtering (e.g., using a bandpass filter), amplifying, inverting, modulating, demodulating, multiplexing, any other suitable signal processing technique or any combination thereof to a signal received from a photodetector.

Step 1304 may include computing one or more parameters based at least in part on a signal received from a photodetector. Computed parameters may include any suitable physiological parameter (e.g., pulse rate, respiration rate), signal metric (e.g., signal to noise ratio, standard deviation), any other suitable parameter, changes in value thereof, or any suitable combination thereof.

Step 1304 may include applying any suitable type of mathematical manipulation of a signal received from a photodetector, database searching, or other suitable computational technique. In some embodiments, step 1304 may include performing a discrete or continuous transform, a statistical operation, a database search, or any other suitable computation or combination thereof based on a signal received from a photodetector. For example, in some embodiments, a patient monitoring system may perform a discrete or continuous wavelet transform on a received signal from a photodetector which may be optically coupled to a patient. The patient monitoring system may compute a respiration rate, pulse rate, or other physiological information, or any changes thereof, based on the output of the wavelet transform.

Step 1306 may include controlling the output of an explicit light source (e.g., an emitter). In some embodiments, step 1306 may include switching on a power supply to an explicit light source, controlling the power delivered to the explicit light source, controlling the intensity of the output of the explicit light source, opening a shutter, redirecting the output of the light source, any other technique for controlling the output of the explicit light source, or any combination thereof.

Shown in FIG. 14 is flow diagram 1400 of illustrative steps for determining physiological information based at least in part on a passive PPG signal from a photodetector in accordance with some embodiments. The illustrative steps of flow diagram 1400 may be performed by a pulse oximeter, which may include one or more PPG sensors.

Step 1402 may include receiving a passive photonic signal at a photodetector of a PPG sensor. The PPG sensor may, but need not, include an emitter configured to optically couple to the photodetector (e.g., provide light which may be attenuated by a patient to the photodetector). The passive photonic signal may be electromagnetic radiation attenuated by a patient. Step 1402 may include using any suitable type of photodetector such as, for example, a photodiode, a photomultiplier tube, a CCD, any other suitable photodetector, or any combination or array thereof to receive the passive photonic signal. In some embodiments, the passive photonic signal of step 1402 may be generated without the use of an emitter. In some embodiments, the passive photonic signal of step 1402 may be generated with an emitter (e.g., an LED) included as a part of the PPG sensor, but the emitter need not be activated.

In some embodiments, step 1402 may include applying signal processing techniques to a passive PPG signal received from a PPG sensor (e.g., an electrical signal). For example, step 1402 may include applying sampling, filtering (e.g., using a bandpass filter), amplifying, inverting, modulating, demodulating, multiplexing, any other suitable signal processing technique or any combination thereof to a passive PPG signal received from a PPG sensor. For example, a pulse oximeter may be configured to receive and process two passive PPG signals from two PPG sensors (e.g., to compute a DPTT value).

Step 1404 may include applying any suitable type of mathematical manipulation of a received passive PPG signal from a photodetector of a PPG sensor, database searching, or other suitable computational technique. In some embodiments, step 1404 may include performing a discrete or continuous transform, applying a correlation, a statistical operation, a database search, any other suitable computation, or combination thereof on a passive PPG signal received from a photodetector. For example, in some embodiments, a patient monitoring system may perform a discrete or continuous wavelet transform on a received passive PPG signal from a photodetector optically coupled to a patient.

Step 1406 may include determining physiological information associated with a patient based at least in part on a received passive PPG signal. Physiological information may include any suitable physiological parameter, or other parameter, such as, for example, pulse rate, respiration rate, any other parameter, changes in value thereof, or any suitable combination thereof. For example, a pulse oximeter may compute a respiration rate, pulse rate, SpO₂, or other physiological information, or any changes thereof, based on the output of a wavelet transform performed in accordance with step 1404.

The illustrative steps of FIGS. 9-14 may be combined, omitted, appended, replaced, rearranged, or otherwise altered in accordance with the present disclosure.

It will also be understood that the previously discussed embodiments and examples are only illustrative of aspects of the disclosed passive sensing, and are not presented for purposes of limitation. It will be understood that various passive sensing techniques may be made available to the user and examples included herein are solely for convenience. Those skilled in the art will appreciate that the disclosed passive sensing systems may be practiced by other than the described embodiments, and the disclosure is limited only by the claims that follow.

What is claimed is:

1. A method for passive physiological sensing, the method comprising:
receiving at least one passive photonic signal from a subject using at least one photodetector; and
determining, using at least one processing device, physiological information of the subject based at least in part on the at least one passive photonic signal.

2. The method of claim 1, further comprising optically filtering the at least one passive photonic signal.

3. The method of claim 1, wherein physiological information comprises a respiration rate, a change in respiration rate, a pulse rate, a change in pulse rate, and/or a SPO2, and/or a combination thereof.

4. The method of claim 1, wherein the receiving at least one photonic signal using at least one photodetector comprises receiving a plurality of photonic signals using a plurality of photodetectors arranged in an array.

5. The method of claim 4, further comprising optically filtering the received photonic signal at one or more photodetectors of the plurality of photodetectors in the array.

6. The method of claim 1, wherein the at least one passive photonic signal from the subject comprises electromagnetic radiation emitted from the subject, electromagnetic radiation transmitted through the subject, and/or electromagnetic radiation reflected from the subject, and/or a combination thereof.

7. The method of claim 1, further comprising:
determining whether to provide active sensing based at least in part on at least one of the passive photonic signal and the physiological information;
activating an explicit light source if it is determined to provide active sensing; and
receiving at least one active photonic signal using the at least one photodetector, wherein the at least one active photonic signal is generated at least in part by the explicit light source.

8. The method of claim 7, wherein the determining whether to provide active sensing is based at least in part on a signal to noise ratio of an output signal from the at least one photodetector.

9. A patient monitoring system comprising:
at least one photodetector configured to receive at least one passive photonic signal;
a signal input configured to receive at least one photodetector signal from the at least one photodetector; and
at least one processing device coupled to the signal input, wherein the at least one processing device is configured to;
determine physiological information based at least in part on the at least one photodetector signal.

10. The system of claim 9, further comprising an explicit light source configured to optically couple to the at least one photodetector.

11. The system of claim 10, wherein the explicit light source is not activated.

12. The system of claim 9, further comprising an explicit light source optically coupled to the at least one photodetector, wherein the explicit light source is activated, and wherein the at least one photonic signal comprises an active photonic signal.

13. The system of claim 9, wherein the at least one processing device is further configured to determine whether to provide active or passive sensing.

14. The system of claim 9, wherein the at least one photodetector comprises a plurality of photodetectors arranged in an array.

15. The system of claim 9, wherein the at least one photodetector comprises a charge coupled detector array.

16. The system of claim 9, further comprising an optical filter optically coupled to the at least one photodetector, wherein the optical filter reduces the intensity of at least some wavelengths of the at least one received photonic signal.

17. A sensor for detecting a photonic signal from a patient, the sensor comprising:
at least one photodetector configured to receive at least one passive photonic signal, wherein the at least one passive photonic signal comprises electromagnetic radiation attenuated by the patient.

18. The sensor of claim 17, further comprising a cable electrically coupled to the detector, wherein the cable is configured to transmit electronic signals from the sensor.

19. The sensor of claim 17, further comprising an explicit light source, wherein the explicit light source is not activated.

20. The sensor of claim 17, further comprising an optical filter coupled to the at least one photodetector, wherein the optical filter reduces the intensity of at least some wavelengths of the at least one received photonic signal.

21. The sensor of claim 17, further comprising a transceiver, a transponder, a transmitter, a receiver, and/or an antenna, and/or a combination thereof configured to wirelessly couple the sensor and a patient monitor.
